(19) United States
(12) Patent Application Publication
(10) Pub. No.: US 2007/0110808 A1
(43) Pub. Date: May 17, 2007

(54) Extended release osmo-microsealed formulation

(75) Inventors: Sampad Bhattacharya, Vadodara (IN); Sridhar Gummudavelli, Vadodara (IN); Mayank Joshi, Vadodara (IN); John S. Egbert, Houston, TX (US)

Correspondence Address: EGBERT LAW OFFICES, 412 MAIN STREET, 7TH FLOOR, HOUSTON, TX, 77002, US

(73) Assignee: ALEMBIC LIMITED, Vadodara (IN)

(21) Appl. No.: 10/563,631

(22) PCT Filed: May 14, 2004
(86) PCT No.: PCT/IN04/00133

§ 371 Date: Jan. 6, 2006

(30) Foreign Application Priority Data
Jun. 5, 2003 (IN) 504/MUM/2002

(51) Int. Cl. A61K 31/137; A61K 9/24

(52) U.S. Cl. 424/473; 514/650

(57) ABSTRACT

The extended release osmo-microsealed formulation includes three controlled release systems associated in series. First, there is an inner solid particulate phase containing Venlafaxine Hydrochloride (Active), and one or more hydrophobic polymers, one or more diluents required to increase the bulk one or more osmogen (agents which can generate osmotic pressure across the hydrophobic coating) and one or more binder polymers essentially to provide strength/hardness to the particle. Second, there is an outer solid continuous phase including one or more hydrophilic polymers, that is further compressed into a tablet. Third, there is an optional functional coat surrounding the tablet. The process/method for forming the Osmo-microsealed extended release delivery system and the process for using such system for treating human ailment/depression are also provided.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The invention relates to extended release delivery system for pharmaceutical such as structurally novel antidepressant venlafaxine hydrochloride active as an 24 hour extended release dosage form. The formulation comprises an inner solid particulate phase containing venlafaxine hydrochloride and one or more hydrophobic polymers, diluents, osmogen and binder polymers, an outer solid continues phase including one or more hydrophilic polymers and compressed into tablets and an functional coat surrounding the tablet optionally provided.

The formulation provides osmo microseal venlafaxine particles and hydrophilic matrix 24 hours extended release dosage form for better control of blood plasma level then the conventional tablet formulation which are administered two or more times a day and there are comparatively lower incidents of nausea and vomiting.

The invention also provides process of preparing osmo microseal extended release delivery system and using such system for treating human ailments such as treatment of depression.

### BACKGROUND OF THE INVENTION

Venlafaxine Hydrochloride, 1-[2-(dimethylamino)-1-(4-methoxyphenyl)ethyl]cyclohexanol Hydrochloride, is an important drug in the neuro-pharmacotherapeutic arsenal used for treatment of depression. Venlafaxine and the acid addition salts thereof are disclosed in U.S. Pat. No. 4,535,186. Venlafaxine hydrochloride is administered in compressed tablet form in doses ranging from 75 to 350 mg/day, in divided doses two or three times a day. With the plural daily dosing regimen, the most common side effect is nausea, experienced by about forty five percent of patients under treatment with Venlafaxine Hydrochloride. Vomiting also occurs in about seventeen percent of the patients. The problem is addressed in European patent 0797 991A1 and U.S. Pat. Nos. 6,274,171, 6,403,120 & 6,419,958 which discloses an extended release once-a-daily pharmaceutical composition (American Home Products, Sherman et. al.; EFFEXOR XRTM) consisting of hard gelatin capsules filled with film coated spheroids comprising a therapeutically effective amount of Venlafaxine Hydrochloride, microcrystalline cellulose and, optionally, Hydroxypropyl methylcellulose extruded and spheronized and the formed spheroids further coated with a mixture of ethyl cellulose and Hydroxypropyl methylcellulose. Venlafaxine has been formulated into a controlled release dosage form with the ability to provide in a single dose a therapeutic blood serum level of the drug over a twenty four hour period. By this method, tighter plasma therapeutic range control can be obtained and a multiple dosing is avoided in this manner. The sharp peaks and troughs in blood plasma drug levels are avoided as well.

With the conventional release dosage forms of Venlafaxine Hydrochloride (tablets), peak blood plasma levels appeared after 2-4 hrs, in contrast to the extended release dosage forms, when plasma levels of Venlafaxine Hydrochloride rose after administration for between five to eight hrs (average—6) and than begin to fall through a protracted, substantially linear decrease from the peak plasma level for the remainder of the period, maintaining therapeutic level of the drug during the entire twenty four hours period. This dosage form when tested in vitro using water at 37?C, 100 rpm and basket has the following dissolution specification,

| Time (hrs) | Mean (% drug dissolved) |
|---|---|
| 2 | <30 |
| 4 | 30-55 |
| 8 | 55-80 |
| 12 | 65-90 |
| 24 | >80 |

In fact, the art acknowledges the difficulty of producing extended release tablets by hydrogel technology because the compressed tablets were either physically unstable (poor compressibility or capping problems) or dissolved too rapidly in dissolution studies.

WO 03/041692 discloses an alternative approach of preparing extended release spheroids of Venlafaxine. Venlafaxine Hydrochloride is coated on a non pareil inert core, which is further coated with an inert polymer layer and subsequently with a third coat of an polymeric layer which enables controlled release.

WO 01/51041 teaches a formulation comprising a tablet and a semi-permeable membrane surrounding the core tablet. The core comprises Venlafaxine and one osmotic agent. The semipermeable membrane surrounding the core has a passageway drilled through it either mechanically or by laser. The coated osmotic drug delivery system based tablet is further coated with an external coat comprising a therapeutically effective amount of an anti-psychotic agent.

WO 98/47491 teaches a novel controlled release composition and the system has been named intelliGITransporters™. The composition can be formulated as an tablet or an suppository and optionally coated with an anionic polymer for enteric effect. The said coat is proposed to prevent the initial burst effect and impart the gastrointestinal tract (GIT) stealth characteristics especially in the presence of food. Prior to coating the core tablet is prepared by mixing a blend of two polymers with opposite wetting characteristics and have a water contact angle theta such that cos of theta is between +0.9848 and −0.9848. Though Venlafaxine is a part of its exhaustive list of the drugs where the proposed technology could be applicable, it does not appear in any of the example.

More recently, WO 03/055475 teaches a composition for once a day administration using hydrogel technology. It describes a process for the preparation of a solid controlled release pharmaceutical formulation comprising the steps of dissolving Venlafaxine and polyvinyl pyrrolidone in an aqueous solvent, applying the resulting solution onto low viscosity hydrophilic polymer, homogeneously mixing the obtained granulate with a high viscosity hydrophilic polymer, and compressing the granulate to obtain a core which is then coated with a polymeric coating comprising a water high permeable polymer and a water low permeable polymer.

### BRIEF SUMMARY OF THE INVENTION

In accordance with the present invention, a novel way has been found of formulating drug with high water solubility such as Venlafaxine Hydrochloride. Briefly, a system is used with the inner phase being an osmotic core comprising a therapeutically effective amount of Active and at least one osmotic agent, a membrane surrounding the core and the outer phase comprising of hydrophilic polymer matrix; the blend is compressed into tablet and subsequently provided a functional coat. The combination of the inner osmo microsealed, hydrophobic core and the outer hydrophilic polymer matrix optionally with a functional coat is claimed to provide for an efficient control and modulation over the release pattern of Venlafaxine Hydrochloride.

For drug with high water solubility such as Venlafaxine Hydrochloride, one of the approach as described in European patent EP0797991 and U.S. Pat. Nos. 6,274,171, 6,403,120 and 6,419,958 is to formulate spheroids of hydrophobic polymers like ethyl cellulose. Though, the process involved in the preparation of spheroids is very tedious as compared to the manufacturing of matrix tablets. In the preferred embodiment of the present invention the core is prepared by the process of granulating admixture of drug, osmogen, diluent and binder with a solution/dispersion of swellable and permeable hydrophobic polymer, and if required, the granulation is followed by coating of the granules with the said hydrophobic polymer. The coating of the granules is achieved by a process known to person of ordinary skill in the said art. The resulting granules can be sifted and resifted to remove any agglomerate produced in the coating steps. In the preferred embodiment, coating may also be achieved by repetitive re-granulation of granulated and subsequently dried mass. The formed internal phase of osmotic core is further admixed with the external phase comprising of hydrophilic polymer(s), lubricants and glidants. This system is compressed into tablets and further provided with a functional coat. The process involved in the preparation of the osmo-microsealed tablets, unlike the manufacturing of spheroids, is very simple and feasible using common equipment. Besides, inclusion of more than one rate-controlling mechanisms in one system provides for a greater control and modulation of the release pattern to achieve desired drug release profile and through it the targeted blood levels.

Some of the polymers used in the preparation of spheroids as well as the osmo-microsealed system are identical, the major difference is in the timing when the core of the present invention and the spheroid described in the prior patents are exposed to the gastrointestinal environment. Spheroids are released immediately into the system following the dissolution of the gelatin shell whereas the exposure of the osmotic core in the current invention is prolonged and regulated by the hydration of the outer hydrophilic matrix. The differential exposure of the core over a period of time provides for reduced requirement of the hydrophobic polymer level in the core and the desired level can be conveniently achieved by the process as simple as granulation. Similarly, the presence of hydrophobic polymer coating over the drug in the preparation of core provides for a reduced level of hydrophilic polymer in the external matrix of the formed tablet. Optionally, the external functional coat provides for achieving the lag phase in the drug release profile.

Best mode for carrying the invention:

A preferred tablet composition comprises:
(i) A hydrophobic core comprised of active ingredient (Venlafaxine hydrochloride), Sodium chloride, Microcrystalline cellulose, Oleic acid, medium chain triglyceride, Povidone K 90 D and Ethyl cellulose.
(ii) A hydrophilic continues phase consisting of Hydroxypropyl methylcellulose, Talc and Magnesium stearate.
(iii) Optionally a functional coat on the compressed tablets consisting Ammonio methacrylate copolymer, Triethyl citrate, Titanium dioxide and color.

### BRIEF DESCRIPTION OF THE SEVERAL VIEWS OF THE DRAWINGS

FIG. 1 is a graph illustration of a plot showing the drug release profile of Venlafaxine Hydrochloride from four different compositions of the drug in matrices using USP I, 100 rpm and at 37 ?C.

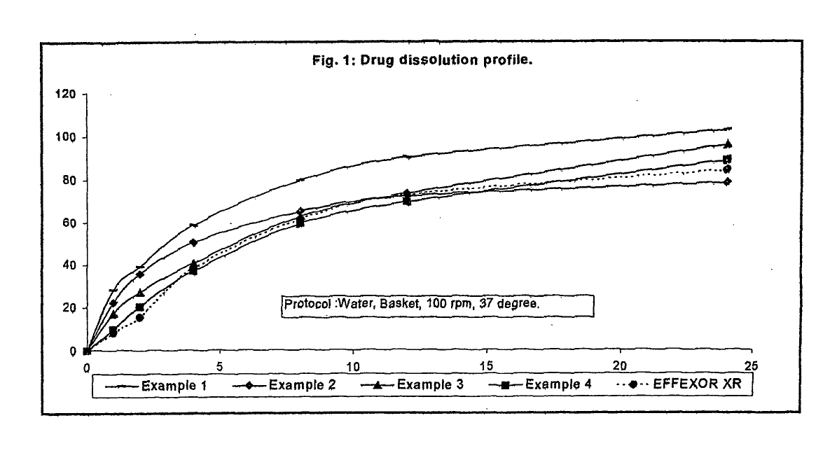

FIG. 2 is a graph illustration of a plot showing the plasma level profile of Venlafaxine Hydrochloride in Healthy Human volunteers.

FIG. 3 is a graph illustration of a plot showing the plasma level profile of O-desmethyl Venlafaxine Hydrochloride in Healthy Human volunteers.

### DETAILED DESCRIPTION OF THE INVENTION

Venlafaxine hydrochloride 1-[2-(dimethylamino)-1 (4methoxyphenyl)ethyl]cyclohexanol hydrochloride is polymorphic. Any of the polymorphic forms may be used in the formulations of the present invention. The invention provides for the administration of Venlafaxine in its free base, free acid, racemic, optically pure, diastereomeric and/or pharmaceutically acceptable salt forms. As used herein, “pharmaceutically acceptable salts” refer to derivatives of the disclosed compounds wherein the therapeutic compound is modified by making acid or base salts thereof. Examples of pharmaceutically acceptable salts include, but are not limited to, non-toxic mineral or organic or inorganic acid salts of venlafaxine. For example, such conventional non-toxic salts include those derived from acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “high water solubility” or similar term when characterizing a drug, medicament or pharmaceutical for use in the formulation of the invention refers to a solubility in water of at least about 50 mg/ml, preferably at least about 100 mg/ml or more, and more preferably greater than 150 mg/ml.

The controlled release system of the invention includes the inner solid particulate phase and the outer solid continuous phase in a weight ratio within the range from about 0.3:1 to about 10:1, preferably from about 0.5:1 to about 4:1. The inner solid particulate phase contain drug in an amount within the range from about 5% to about 75% by weight, preferably from about 7% to about 65% by weight, a hydrophobic polymer in an amount within the range from about 0.5% to about 65% by weight, preferably from about 2% to about 45% by weight, an osmogen in the range from about 0.01% to about 25% by weight, preferably from 0.05% to about 10% by weight, a binder to provide strength/hardness to the particle in the range from about 0.1% to about 10% by weight, preferably from 0.5% to about 8% by weight and it may contain a pharmaceutical diluent(s) in an amount within the range from about 0% to about 90% by weight, preferably from 20% to about 80% by weight, the above percentages being based on the weight of the inner solid particulate phase.

The inner solid particulate phase have a mean particle size within the range from about 0.01 micrometer to about 2 mm, and preferably from about 50 micrometer to about 0.5 mm.

The outer continuous phase may contain one or more hydrophilic polymers in the range from about 3% to about 60% by weight and preferably from about 10% to about 55% by weight. Besides, the outer continuous phase in the various formulation of the invention may optionally include one or more fillers or excipients in an amount within the range from about 1% to about 70% by weight and more preferably 10% to about 40% by weight, the above percentages being based on the weight of the uncoated dosage form. The uncoated dosage form also contains in the outer continous phase the recommended level of glidants, lubricants, dry binders and anti-adherents.

The dosage of the invention is coated as is commonly done in the art to provide the desired functional property. The coating may comprise from about 2 to about 20% by weight, preferably from 2.5 to 10% by weight of the uncoated tablet core.

The hydrophobic polymer(s) insoluble in the liquids of the gastrointestinal tract, which may be employed in the inner solid particulate phase includes by way of example and without limitation, ethyl cellulose, methyl cellulose, amino methacrylate copolymer, methacrylic acid copolymers, methacrylic acid acrylic acid ethyl ester copolymer, methacrylic acid esters neutral copolymer, dimethyl aminoethyl methacrylate-methacrylic acid esters copolymer, Cellulose acetate, vinyl methyl ether/ maleic anhydride copolymers. The hydrophobic polymer is suitable for use in the form of a Non aqueous solution, aqueous suspension, an aqueous emulsion, or a water-containing organic solvent solution. They are also commercially available as, for example, Eudragit L 30D, Eudragit E30D, Aquacoat ECD-30, Surelease E-7, Eudragit RS 30D, Eudragit NE 30D, Eudragit RL 30D, etc.

Exemplary osmagens include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, sorbitol, combinations thereof and other similar or equivalent materials which are widely known in the art.

As used herein, the term “diluents” and “fillers” is intended to mean inert substances used as excipients to create the desired bulk, flow properties, and compression characteristics in the preparation of tablet. Such compounds include, by way of example and without limitation, dibasic calcium phosphate, kaolin, lactose, sucrose, mannitol, microcrystalline cellulose, powdered cellulose, precipitated calcium carbonate, sorbitol, and starch and other materials known to one of ordinary skill in the art.

The binder(s) used essentially to provide strength/hardness, which may be employed in the inner solid particulate phase, includes by way of example and without limitation, polyacryl amide, poly-N-vinyl amide, poly-N-vinyl-acetamide, polyvinyl pyrolidone, starch, lactose, modified corn starch, sugars, gum accacia, alginic acid, carboxymethylcellulose sodium, tragacanth, gelatin, liquid glucose, methylcellulose, pregelatinized starch, polyethylene glycol, guar gum, polysaccharide, bentonites, invert sugars, collagen, albumin, polypropylene glycol, polyoxyethylene-polypropylene copolymer, polyethylene ester, polyethylene sorbitan ester, polyethylene oxide, and hydroxypropyl methylcellulose, combinations thereof and other materials known to one of ordinary skill in the art. Important characteristics of suitable Hydroxypropyl methylcelluloses include a low viscosity, preferably less than 10 Cps and more preferably 2 to 5 Cps. Other equivalents of the Hydroxypropyl methylcelluloses 2208 and 2910 USP, having the same chemical and physical characteristics as the proprietary products named above may be substituted in the formulation.

The hydrophilic polymer(s) in the outer continuous phase includes by way of example and without limitation, hydroxyethyl cellulose, hydroxypropyl cellulose, sodium alginate, carbomer (Carbopol™), sodium carboxymethyl cellulose, xanthan gum, guar gum, locust bean gum, poly vinyl acetate, polyvinyl alcohol and hydroxypropyl methylcellulose.

The functional coating layer which is optionally applied over the outer solid phase containing particles of the inner solid phase embedded therein may include one or more film-formers, such as the polymer like methacrylic acid esters neutral polymer, ethyl cellulose, cellulose acetate, polyvinyl alcohol-maleic anhydride copolymers, beta-pinene polymers, glyceryl esters of wood resins and the like. Both core tablets as well as coating formulations may contain aluminium lakes to provide color. Even the commercially available dispersion of film formers namely, Opadry, Eudragit L 30D, Eudragit E30D, Aquacoat ECD-30, Surelease E-7, Eudragit RS 30D, Eudragit NE 30D, Eudragit RL 30D, etc. may be used for the purpose of providing functional coat.

The film formers both in the inner particulate phase and on the outer continuous phase may be applied form a solvent system containing one or more solvents including water, ammonium hydroxide solution, sodium hydroxide solution, hydrochloric acid solution, alcohols like methyl alcohol, ethyl alcohol or isopropyl alcohol, ketones like acetone, or ehtylmethyl ketone, chlorinaed hydrocarbons like methylene chloride, dichloroethane, and 1,1,1-trichloroethane.

Plasticizers can also be included in the dosage form to modify the properties and characteristics of the polymers used in the coats of inner particulate phase and/or on the coat of the compressed tablet. Plasticizers useful in the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. It is also contemplated and within the scope of the invention, that a combination of plasticizers may be used in the present formulation.

The dosage form of the invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides, medium chain triglycerides and acetylated fatty acid glycerides.

The dosage form of the invention can also comprise an antiadherent, glidant, lubricant, opaquant, colorant, polishing agents, acidifying agent, alkalizing agent, antioxidant, buffering agent and surface active agent.

Antiadherents include, by way of example and without limitation, magnesium stearate, talc, calcium stearate, glyceryl behenate, Polyethylene glycols, hydrogenated vegetable oil, mineral oil, stearic acid and other materials known to one of ordinary skill in the art.

Glidants include, by way of example and without limitation, cornstarch, talc, calcium silicate, magnesium silicate, colloidal silicon dioxide, silicon hydrogel and other materials known to one of ordinary skill in the art.

Lubricants include, by way of example and without limitation, calcium stearate, magnesium stearate, mineral oil, stearic acid, and zinc stearate and other materials known to one of ordinary skill in the art.

Opaquant may be used alone or in combination with a colorant. Such compounds include, by way of example and without limitation, titanium dioxide and other materials known to one of ordinary skill in the art.

Colorant include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other F.D. & C. dyes and natural coloring agents such as grape skin extract, beet red powder, beta-carotene, annato, carmine, turmeric, paprika, and other materials known to one of ordinary skill in the art. The amount of coloring agent used will vary as desired.

Polishing agents include, by way of example and without limitation, camauba wax, and white wax and other materials known to one of ordinary skill in the art.

Acidifying agents include, by way of example and without limitation, acetic acid, amino acid, citric acid, fumaric acid and other alpha hydroxy acids, such as hydrochloric acid, ascorbic acid, and nitric acid and others known to those of ordinary skill in the art.

Alkalizing agents include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art.

Antioxidants include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art.

Buffering agents include, by way of example and without limitation, potassium metaphosphate, potassium phosphate, monobasic sodium acetate and sodium citrate anhydrous and dihydrate and other materials known to one of ordinary skill in the art.

The present dosage form can also employ one or more commonly known surface active agents that improve wetting of the tablet core or layers. Soaps and synthetic detergents may be employed as surfactants and as vehicles for detergent compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl.beta.-aminopropionates and 2-alkylimidazoline quaternary ammonium salts; and mixtures thereof.

It should be understood, that compounds used in the art of pharmaceutical formulation generally serve a variety of functions or purposes. Thus, if a compound named herein is mentioned only once or is used to define more than one term herein, its purpose or function should not be construed as being limited solely to that named purpose(s) or function(s).

The following examples should not be considered exhaustive, but merely illustrative of only a few of the many embodiments contemplated by the present invention. The methods described herein can be followed to prepare osmo-microsealed devices according to the invention.

Examples 1 to Example 4 illustrates the development sequence to arrive at the said extended release dosage form. The composition for Example 1 to 4 is recorded in Table 1.

### EXAMPLE 1

Mix Venlafaxine Hydrochloride and Microcrystalline cellulose in rapid mixer granulator for 15.0 minutes. Prepare the binder liquid by dissolving Polyvinyl Pyrolidone in the required quantity of Water with stirring. Granulate the mass and mix for 10.0 minutes. Dry the above granules in a fluid bed drier and size it through a multi mill. Lubricate the sifted granules with Hydroxypropyl Methylcellulose, Talc and Magnesium stearate in a cone blender. Prepare tablets by compressing the above blend.

### EXAMPLE 2

Mix Venlafaxine Hydrochloride, Microcrystalline Cellulose and Polyvinyl Pyrolidone in cone blender for 20.0 minutes. Granulate the blend with an aqueous dispersion of ethyl cellulose containing Oleic acid and medium chain triglyceride in a solution of ammonium hydroxide (Surelease E-7). Dry the granules and size it using multi mill. Lubricate the sifted granules with Hydroxypropyl Methylcellulose, Talc and Magnesium stearate in a cone blender. Prepare tablets by compressing the above blend.

### EXAMPLE 3

Mix Venlafaxine Hydrochloride, Microcrystalline Cellulose and Polyvinyl Pyrolidone in cone blender for 20.0 minutes. Granulate the blend with an aqueous solution of Sodium chloride in a fluid bed processor. Continue the granulation with an aqueous dispersion of ethyl cellulose containing Oleic acid and medium chain triglyceride in a solution of ammonium hydroxide (Surelease E-7). Dry the granules and size it using multi mill. Lubricate the sifted granules with Hydroxypropyl Methylcellulose, Talc and Magnesium stearate in a cone blender. Prepare tablets by compressing the above blend.

### EXAMPLE 4

Mix Venlafaxine Hydrochloride, Microcrystalline Cellulose and Polyvinyl Pyrolidone in cone blender for 20.0 minutes. Granulate the blend with an aqueous solution of Sodium chloride in a fluid bed processor. Continue the granulation with an aqueous dispersion of ethyl cellulose containing Oleic acid and medium chain triglyceride in a solution of ammonium hydroxide (Surelease E-7). Dry the granules and size it using multi mill. Lubricate the sifted granules with Hydroxypropyl Methylcellulose, Talc and Magnesium stearate in a cone blender. Prepare tablets by compressing the above blend. Coat the tablet with an aqueous dispersion of amino methacrylate copolymer containing Triethyl citrate, Talc and Titanium dioxide.

The composition for Example 5 to 12 is recorded in Table 2, which illustrates the various combinations, and the processes, which can be used to prepare the claimed dosage form.

### EXAMPLE 5

Mix Venlafaxine Hydrochloride, Copolyvidone, Lactose and Mannitol in RMG for 15.0 minutes. Prepare the film forming liquid by dissolving Cellulose acetate and polyethylene glycol into the required quantity of Dichloromethane: Isopropyl alcohol (2:1) with stirring. Granulate the mass with partial quantity of the film forming liquid and mix for 30.0 minutes. Dry the above granules in a fluid bed drier. Re-granulate the mass with the remaining quantity of the film forming liquid. Repeat the process and dry the granules to achieve the desired film coating of the granules. Size the granules using multi mill. Lubricate the sifted granules with Carbomer, Dibasic Calcium Phosphate and Glyceryl behenate in a cone blender. Compress the above blend into tablets and coat them with a freshly prepared aqueous dispersion of Eudragit RS, Triethyl citrate, Talc and Titanium dioxide in water.

### EXAMPLE 6

Mix Venlafaxine Hydrochloride, Lactose and Mannitol in cone blender. Prepare the film forming liquid by dispersing Cellulose acetate and polyethylene glycol into the required quantity of Dichloromethane: Isopropyl alcohol (2:1) with stirring. Granulate the blended mass with an aqueous solution of Copolyvidone in a fluid bed processor. Continue the granulation with the film forming solution in a fluid bed processor. Size the granules using multi mill. Lubricate the granules with Carbomer, Dibasic Calcium Phosphate and Glyceryl behenate in a cone blender. Compress the above blend into tablets and coat them with a freshly prepared dispersion of Eudragit RS, Triethyl citrate, Talc and Titanium dioxide in water.

### EXAMPLE 7

Mix Venlafaxine Hydrochloride, Lactose and Mannitol in cone blender for 8.0. minutes. Granulate the mass with an aqueous solution of Hydroxypropyl Methylcellulose (Methocel E3) in a fluid bed processor. Prepare the film forming liquid by dissolving Cellulose acetate and polyethylene glycol into the required quantity of Dichloromethane: Isopropyl alcohol (2:1) with stirring. Coat the dried granules with the film forming solution in a Wurster fluid bed processor. Size the granules using multi mill. Lubricate the coated granules with Hydroxypropyl Methylcellulose, Dibasic Calcium Phosphate, Magnesium Stearate and Talc in a cone blender. Compress the above blend into tablets and coat them with a freshly prepared dispersion of Eudragit RS, Triethyl citrate, Talc and Titanium dioxide in water.

### EXAMPLE 8

Mix Venlafaxine Hydrochloride, Microcrystalline Cellulose and Lactose in RMG for 20.0 minutes. Granulate the mass with an aqueous solution of Sodium chloride. Dry the granules in a fluid bed drier. Coat the dried granules in a Wurster fluid bed processor with the aqueous dispersion of ethyl cellulose containing Oleic acid and medium chain triglyceride in aqueous solution of ammonium hydroxide (Surelease E 7). Size the granules using multi mill. Lubricate the sifted granules with Hydroxypropyl Methylcellulose, Carbomer 934 P, Magnesium Stearate and Talc in a cone blender. Compress the above blend into tablets and coat them with a freshly prepared dispersion of Eudragit RL, Triethyl citrate, Talc and Titanium dioxide in water.

### EXAMPLE 9

Mix Venlafaxine Hydrochloride and Mannitol in RMG for 5.0 minutes. Granulate the mass with an aqueous solution of Povidone. Dry the granules in a fluid bed drier. Prepare the film forming liquid by dispersing Cellulose acetate and polyethylene glycol into the required quantity of Dichloromethane: Isopropyl alcohol (2:1) with stirring. Coat the dried granules with the film forming solution in a Wurster fluid bed processor. Lubricate the coated granules with Hydroxypropyl Methylcellulose, Dibasic Calcium Phosphate, Magnesium stearate and Talc in a cone blender. Compress the above blend into tablets and coat them with a freshly prepared dispersion of Eudragit RL, Eudragit RS, Triethyl citrate, Talc and Titanium dioxide in water.

### EXAMPLE 10

Mix Venlafaxine Hydrochloride, sodium chloride and Microcrystalline Cellulose in RMG for 5.0 minutes. Granulate the mass with an aqueous solution of Povidone. Dry the granules in a fluid bed drier. Prepare the film forming liquid by dispersing Cellulose acetate and polyethylene glycol into the required quantity of Dichloromethane: Isopropyl alcohol (2:1) with stirring. Coat the dried granules with the film forming solution in a Wurster fluid bed processor. Lubricate the coated granules with Hydroxypropyl Methylcellulose, Dibasic Calcium Phosphate and glyceryl behenate in a cone blender. Compress the above blend into tablets and coat them with a freshly prepared dispersion of Eudragit RL, Eudragit RS, Triethyl citrate, Talc and Titanium dioxide in water.

### BIOAVAILABILITY STUDIES

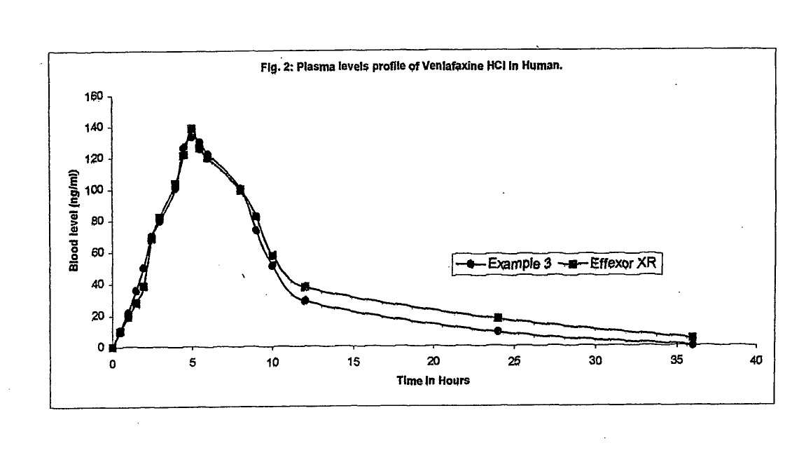

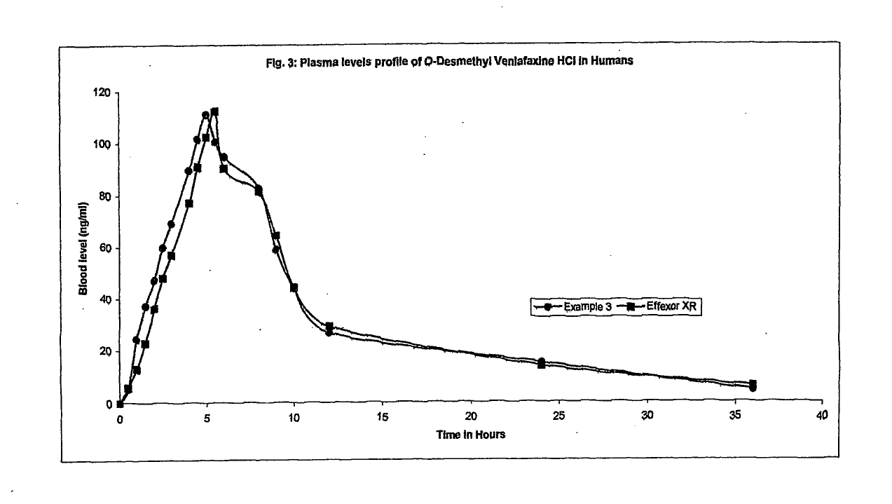

A randomized, two-treatment, two-period, two-sequence, single dose, crossover bioavailability study on Venlafaxine 150 mg extended release tablets (Example 3), compared with Venlafaxine 150 mg extended release capsule (Effexor XRTM) manufactured by Wyeth-Ayerst, in six, healthy, adult, male, human subjects was conducted under non fasting conditions. The extended release plasma level profile of Venlafaxine and its Active metabolite O-desmethyl Venlafaxine is demonstrated in FIG. 2 and FIG. 3 respectively.

TABLE 1

| | | Percentage w/w | | | |
|---|---|---|---|---|---|
| Sr. No. | EXAMPLE | 1 | 2 | 3 | 4 |
| 1 | Venlafaxine Hydrochloride | 21.38 | 21.38 | 21.38 | 19.44 |
| 2 | Microcrystalline cellulose | 17.75 | 17.75 | 16.25 | 14.77 |
| 3 | Polyvinyl pyrolidone | 1.66 | 1.66 | 1.66 | 1.51 |
| 4 | Sodium Chloride | — | — | 1.50 | 1.36 |
| 5 | Medium Chain Triglycerides | — | 0.86 | 0.86 | 0.78 |
| 6 | Ethyl Cellulose | — | 13.85 | 13.85 | 12.59 |
| 7 | Oleic Acid | — | 1.75 | 1.75 | 1.59 |
| 8 | Ammonium Hydroxide (28%) | — | Lost in | Lost in | Lost in |
| | | | Processing | Processing | Processing |
| 9 | Purified Water | Lost in | Lost in | Lost in | Lost in |
| | | Processing | Processing | Processing | Processing |
| 10 | Hydroxypropyl Methylcellulose | 57.71 | 41.25 | 41.25 | 37.50 |
| 11 | Magnesium Stearate | 1 | 1 | 1 | 0.91 |
| 12 | Talc | 0.5 | 0.5 | 0.5 | 1.98 |
| 13 | Trimethyl amino methacrylate | — | — | — | 5.35 |
| | copolymer, Type A | | | | |
| 14 | Triethyl citrate | — | — | — | 1.07 |
| 15 | Titanium dioxide | — | — | — | 1.14 |

TABLE 2

| | | Percentage w/w | | | | |
|---|---|---|---|---|---|---|
| Sr. No. | EXAMPLE | 5-6 | 7 | 8 | 9 | 10 |
| 1 | Venlafaxine Hydrochloride | 39.06 | 38.46 | 20.46 | 21.25 | 22.70 |
| 2 | Microcrystalline cellulose | — | — | 8.00 | — | 13.3 |
| 3 | Lactose | 17.47 | 17.07 | 10.49 | — | — |
| 4 | Polyvinyl pyrolidone | — | — | — | 3.13 | 2.67 |
| 5 | Copolyvidone | 2.44 | — | — | — | — |
| 6 | Hydroxypropyl methylcellulose | — | 1.22 | — | — | — |
| 7 | Sodium Chloride | — | — | 0.66 | — | 4.00 |
| 8 | Mannitol | 3.66 | 1.22 | — | 1.25 | — |
| 9 | Medium Chain Triglycerides | — | — | 0.24 | — | — |
| 10 | Ethyl Cellulose | — | — | 5.88 | — | — |
| 11 | Cellulose acetate | 8.38 | 5.25 | — | 17.67 | 8.63 |
| 12 | Oleic Acid | — | — | 0.49 | — | — |
| 13 | Polyethylene glycol | 1.46 | 0.73 | — | 0.25 | 1.1 |
| 14 | Ammonium Hydroxide (28%) | — | — | Lost in | — | — |
| | | | | Processing | | |
| 15 | Purified Water/Isopropyl | Lost in | Lost in | — | Lost in | Lost in |
| | alcohol/Dichloro methane | Processing | Processing | | Processing | Processing |
| 16 | Hydroxypropyl Methylcellulose | — | 17.07 | 29.63 | 30.88 | — |
| 17 | Carbomer | 12.20 | — | 4.32 | — | 23.63 |
| 18 | Dibasic Calcium Phosphate | 10.73 | 11.46 | — | 12.50 | 16.90 |
| 19 | Magnesium Stearate | — | 1.46 | 1.95 | 0.63 | — |
| 20 | Glyceryl behenate | 1.22 | — | — | — | 2.00 |
| 21 | Talc | 0.55 | 1.82 | 3.75 | 2.55 | 0.83 |
| 22 | Trimethyl amino methacrylate | — | — | 10.0 | 3.00 | 2.00 |
| | copolymer, Eudragit RL | | | | | |
| 23 | Trimethyl amino methacrylate | 2.00 | 3.00 | — | 4.00 | 1.00 |
| | copolymer, Eudragit RS | | | | | |
| 24 | Triethyl citrate | 0.40 | 0.60 | 2.00 | 1.40 | 0.60 |
| 25 | Titanium dioxide | 0.43 | 0.64 | 2.13 | 1.49 | 0.64 |

We claim:

1. An extended release osmo microsealed formulation comprising:
an inner solid osmo-microsealed particulate phase being comprised of a therapeutically effective amount of venlafaxine Active or salt thereof and at least one osmogen/osmotic agent or osmo polymer, a diluent, a binder and a hydrophobic polymer membrane forming the core; and
an outer solid continuous phase being comprised of a hydrophilic water soluble and/or swellable polymer, compressed into tablets and optionally coated with a functional coat.

2. The formulation of claim 1, wherein the inner osmo microsealed particulate phase and the outer continuous phase is in a ratio within the range of 0.3:1 to 10:1, preferably from 0.5:1 to about 4:1.

3. The formulation of claim 1, wherein the inner solid particulate phase is comprised of active drug or salt there of in an amount within the range from about 5% to 75%, preferably from about 7% to 65% by weight, ethyl cellulose and/or cellulose acetate in an amount within the range from 0.5% to 65% by weight, preferably from 2% to 45% by weight sodium chloride and/or mannitol in the range from 0.01% to 25% by weight, preferably from 0.05% to 10% by weight, Polyvinyl pyrolidone and/or Hydroxypropyl methylcellulose (low viscosity) in the range from 0.1% to 10% by weight, preferably from 0.5% to 8% by weight; and wherein the inner solid particulate phase is further comprised of microcrystalline cellulose and/or lactose in an amount within the range from about 0% to 90% by weight, preferably from 20% to 80% by weight, the above percentages being based on the weight of the inner solid particulate phase, wherein binding provided by diluents like lactose is sufficient, a specialty binder being excluded.

4. The formulation of claim 1, wherein the inner solid particulate phase is further comprised of a hydrophobic polymer in an amount within the range from about 0.5% to 65% by wt. preferable from about 2% to 45% by wt. of the inner solid particulate phase.

5. The formulation of claim 4, wherein the hydrophobic polymer is used in the form of a non-aqueous solution, aqueous suspension, an aqueous emulsion or a water containing organic solvent solution.

6. The formulation of claim 4, wherein the hydrophobic polymer is selected from a group consisting of: ethyl cellulose, methyl cellulose, amino methacrylate copolymer, methacrylic acid copolymers, methacrylic acid acrylic acid ethyl ester copolymer, methacrylic acid esters neutral copolymer, dimethyl aminoethyl methacrylate-methacrylic acid esters copolymer, Cellulose acetate, vinyl methyl ether/maleic anhydride copolymers.

7. The formulation of claim 1, wherein the inner solid particulate phase is further comprised of an osmogen in an amount within the range from about 0.01% to about 25% by wt. preferably from 0.05% to about 10% by wt. of the inner solid particulate phase.

8. The formulation of claim 7, wherein the osmogens are selected from a group consisting of: organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, sorbitol and the other similar or equivalent materials and combination thereof.

9. The formulation of claim 1, wherein the inner solid particulate phase is further comprised of a binder in the range from about 0.1% to about 10% by wt. preferably from 0.5% to about 8% by wt of the inner solid particulate phase.

10. The formulation of claim 9, wherein the binder is selected from a group consisting of: polyacryl amide, poly-N-vinyl amide, poly-N-vinyl-acetamide, polyvinyl pyrolidone, starch, lactose, modified corn starch, sugars, gum accacia, alginic acid, carboxymethylcellulose sodium, tragacanth, gelatin, liquid glucose, methylcellulose, pregelatinized starch, polyethylene glycol, guar gum, polysaccharide, bentonites, invert sugars, collagen, albumin, polypropylene glycol, polyoxyethylene-polypropylene copolymer, polyethylene ester, polyethylene sorbitan ester, polyethylene oxide, and hydroxypropyl methylcellulose and the other similar or equivalent materials or combination thereof.

11. The formulation of claim 10, wherein the viscosity of hydroxypropyl methylcellulose are of low viscosity preferably less than 10 Cps and more preferably 2 to 5 Cps.

12. The formulation of claim 1, wherein the inner solid particulate phase is further comprised of a diluent in an amount within the range from about 0 to 90% by wt or preferably from about 20% to 80% by wt of the inner solid particulate phase.

13. The formulation of claim 12, wherein the diluent is an inert substance used as excipients to create the desired bulk flow properties and compression characteristic required in the preparation of tablets.

14. The formulation of claim 12, wherein the diluent is selected from a groups consisting of: dibasic calcium phosphate, kaolin, lactose, sucrose, mannitol, microcrystalline cellulose, powdered cellulose, precipitated calcium carbonate, sorbitol, and starch and the like materials.

15. The formulation of claim 1, wherein the inner solid particulate phase has a mean particle size within the range from about 0.01 micrometer to about 2mm, and preferably from about 50 micrometer to about 0.5 mm.

16. The formulation of claim 1, wherein said outer solid continuous phase is further comprised of hydrophilic polymers in an amount within the range from about 3% to 60% by wt and preferably from about 10% to 55% by wt of the uncoated dosage form/tablet.

17. The formulation of claim 16, wherein the hydrophilic polymer is selected from a groups consisting of: hydroxyethyl cellulose, hydroxypropyl cellulose, sodium alginate, carbomer (Carbopol™), sodium carboxymethyl cellulose, xanthan gum, guar gum, locust bean gum, poly vinyl acetate, polyvinyl alcohol and hydroxypropyl methylcellulose.

18. The formulation of claim 16, wherein said outer solid continuous phase is further comprised of one or more fillers or excipients in an amount within the range from about 1% to 70% by wt. and more preferably 10% to 40% by wt. of the uncoated dosage form/tablet.

19. The formulation of claim 16, wherein said outer solid continuous phase is further comprised of a recommended level of glidants, lubricants, dry binders, anti adherents.

20. The formulation of claim 1, wherein the functional coat provided optionally is about 2% to 20% by wt. preferably from 2.5% to 10% by wt. of the uncoated tablet core.

21. The formulation of claim 20, wherein the functional coating layer optionally provided over the outer solid continous phase containing particulates of inner solid phase embedded therein, is further comprised of one ore more film formers such as methacrylic acid esters neutral polymer, ethyl cellulose, cellulose acetate, polyvinyl alcohol-maleic anhydride copolymers, beta-pinene polymers, glyceryl esters of wood resins and the like.

22. The formulation of claim 20, further comprising:
a suitable colouring agent added in the coating.

23. The formulation of claim 1, further comprising:
plastizers to modify the properties and characteristic of polymers used in the coats of inner particulate phase and/or on the coat of compressed tablets.

24. The formulation of claim 23, wherein the plastizers are selected from a group consisting of: low molecular wt polymers, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate or combination thereof.

25. The formulation of claim 24, wherein oils used are selected from a group consisting of: peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides, medium chain triglycerides and acetylated fatty acid glycerides.

26. The formulation of claim 1, wherein the dosage form/tablet is further comprised of antiadherent, glidant, lubricant, opaquant, colorant, polishing agents, acidifying agent, alkalizing agent, antioxidant, buffering agent and surface active agent.

27. The formulation of claim 26, wherein the antiadherent are selected from a group consisting of magnesium stearate, talc, calcium stearate, glyceryl behenate, Polyethylene glycols, hydrogenated vegetable oil, mineral oil, stearic acid and the like materials.

28. The formulation of claim 26, wherein the glidant are selected from a group consisting of cornstarch, talc, calcium silicate, magnesium silicate, colloidal silicon dioxide, silicon hydrogel and the like materials.

29. The formulation of claim 26, wherein the lubricant are selected from a group consisting of: calcium stearate, magnesium stearate, mineral oil, stearic acid, and zinc stearate and the like materials.

30. The formulation of claim 26, wherein opaquant is used alone or in combination with colorant such as titanium dioxide and the like materials.

31. The formulation of claim 26, wherein the colorant are selected from a group consisting of: FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other F.D. & C. dyes and natural coloring agents such as grape skin extract, beet red powder, beta-carotene, annato, carmine, turmeric, paprika and the like materials.

32. The formulation of claim 26, wherein the polishing agent are selected from a group consisting of camauba wax, white wax and the like materials.

33. The formulation of claim 26, wherein the acidifying agent are selected from a group consisting of acetic acid, amino acid, citric acid, fumaric acid and other alpha hydroxy acids, such as hydrochloric acid, ascorbic acid, and nitric acid and the like materials.

34. The formulation of claim 26, wherein the alkalizing agent are selected from a group consisting of: ammonia solution, ammonium carbonate, diethanolamine, monoethanolamine, potassium hydroxide, sodium borate, sodium carbonate, sodium bicarbonate, sodium hydroxide, triethanolamine, and trolamine and the like materials.

35. The formulation of claim 26, wherein the antioxidants are selected from a group consisting of: ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and the like materials.

36. The formulation of claim 26, wherein the buffering agent are selected from a group consisting of: potassium metaphosphate, potassium phosphate, monobasic sodium acetate and sodium citrate anhydrous and dehydrate and the like materials.

37. The formulation of claim 1, further comprising:
a dosage form/tablet having surfaces active agent that improve wetting of the tablet core or coating layers.

38. The formulation of claim 37, wherein the surface active agent are soaps and synthetic detergents.

39. The formulation of claim 38, wherein the soaps are further comprised of fatty acid alkali metal, ammonium, and triethanolamine salts.

40. The formulation of claim 38, wherein the detergents are cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl.beta.-aminopropionates and 2-alkylimidazoline quaternary ammonium salts; and mixtures thereof.

41. A process of preparing an extended release osmo-microsealed formulation comprising the following steps:
forming osmo microsealed inner solid particulate phase by granulation of venlafaxin active or salt thereof with one or more diluents to increase the bulk, binder to provide strength/hardness to the particulate one or more osmogen for generating osmatic pressure across the hydrpobic coating and hydrophobic polymer;
embedding the inner solid particulate phase in an outer solid continous phase being comprised of one or more hydrophilic polymers;
compressing the biphasic blend into tablet; and
coating the tablet optionally with a functional coat containing polymers.

42. A process as claimed in claim 41, wherein the inner osmo microsealed particle coat is obtained by granulation of drug, diluent, binder and osmogen mixture with the dispersion of the coating polymer forming a matrix of drug, diluent, osmogen and the polymer, the granules being re-granulated till the entire coat is applied if required.

43. A process as claimed in claim 41, wherein the inner osmo microsealed particle coat is obtained by granulation of drug, diluent and binder with the solution of osmogen, said granulation being further continued with the dispersion of hydrophobic polymer, the granules being re-granulated till the entire coat is applied if required.

44. A process as claimed in claim 41, wherein the inner osmo microsealed particle coat is obtained by granulation of drug, diluent and osmogen with the solution of binder, said granulation being further continued with the dispersion of hydrophobic polymer, the granules being re-granulated till the entire coat is applied if required.

45. A process as claimed in claim 41, wherein the inner osmo microsealed particle coat is obtained by partial granulation of the drug, diluent and osmogen mixture with the dispersion of coating polymer forming a matrix of drug, diluent, osmogen and the polymer, said granules being further coated on a fluid bed processor with the remaining quantity of the hydrophobic polymer.

46. A process as claimed in claim 41, wherein the inner osmo microsealed particle are obtained by granulation of the drug, osmogen and binder, said granules being coated on a fluid bed processor with the hydrophobic coating polymer.

47. A process as claimed in claim 41, wherein the inner osmo microsealed particle are obtained by granulation of the drug, binder and diluent using a solution of osmogen, said granules being further coated on a fluid bed processor with the hydrophobic coating polymer

48. A process as claimed in claim 41, wherein the inner osmo microsealed particle are obtained by extrusion-spheronization of wet blended mass of drug, binder, diluent and osmogen, mini spherules being obtained and coated on a fluid bed processor with the hydrophobic coating polymer.

49. (canceled)

50. (canceled)

51. (canceled)

52. (canceled)

53. (canceled)

54. (canceled)

55. (canceled)

56. (canceled)
